(19) United States
(12) Patent Application Publication
(10) Pub. No.: US 2026/0272072 A1
(43) Pub. Date: Sep. 17, 2026

(54) Detachable Integration of Dual Moisture-Wicking and Absorbent Sweat Strips for Compression Sleeve

(71) Applicants: Rita BROWN-HOLLOWAY, Orange, CT (US); Tyrone HOLLOWAY, Orange, CT (US)

(72) Inventors: Rita BROWN-HOLLOWAY, Orange, CT (US); Tyrone HOLLOWAY, Orange, CT (US)

(21) Appl. No.: 19/677,231

(22) Filed: May 14, 2026

Related U.S. Application Data
(63) Continuation-in-part of application No. 18/754,446, filed on Jun. 26, 2024.

(51) Int. Cl. A41D 13/00; A41D 13/08; A41D 19/00; A41D 31/12; A41D 31/18

(52) U.S. Cl. CPC A41D 13/0002; A41D 13/08; A41D 19/0017; A41D 31/12; A41D 31/18

(57) ABSTRACT

A compression sleeve system that allows athletic wear to achieve certain perspiration management and comfort requirements, while simultaneously achieving maximum possible sweat control utilizing detachable dual moisture-wicking and absorbent sweat strips to improve moisture absorption and evaporation performance with compression therapy and moisture management protocols in sports and exercise environments.

### BACKGROUND

### 1. Field of the Invention

The present disclosure pertains to athletic wear designed for physical activities. The system includes compression sleeves combined with detachable dual moisture-wicking and absorbent sweat strips to improve sweat management and offer enhanced comfort during exercise.

### 2. Description of Related Art

In the following paragraphs, an overview of existing wearable sweat-management accessories for athletes engaged in vigorous physical activity is provided. Athletes commonly experience profuse sweating, which, while important for thermoregulation, can create distractions, impair vision, and pose safety hazards on the field or court. Traditional headbands and wristbands rely on absorbent materials to collect perspiration in targeted areas; however, these conventional accessories often fail to provide thorough coverage and become rapidly saturated, requiring frequent replacement during extended periods of play. As a result, athletes and equipment managers have sought alternative solutions to address the limitations of these basic sweat-control garments.

Accordingly, to expand the scope of sweat control beyond the forehead and wrists, some athletes resort to carrying towels or attaching larger absorbent panels to their gear. While these approaches can extend moisture management, they tend to be bulky and disrupt the natural flow of competition. Compression sleeves have gained popularity for their purported benefits in circulation enhancement, muscle support, and thermal regulation. As standalone garments, however, they offer no dedicated mechanism for sweat collection, often requiring athletes to juggle multiple accessories or pause activity to manage excess moisture.

Several attempts have been made to address these shortcomings. For example, U.S. Pat. No. 9,936,751 discloses a sweat absorption material designed to cover an athlete's entire forearm or to be worn over a compression sleeve. However, this configuration does not provide the benefits of compression and results in a bulky, movement-restrictive garment that can impede athletic performance and increase heat retention. The arm sleeve described therein may also slide over the smooth surface of a compression sleeve, leading to instability during play.

Another configuration, U.S. Patent Application Publication No. 2005/0049491A1, describes a fingerless glove with a pocket for a thick, foam pad intended to protect the palm following surgery. This device is not designed for sweat management, lacks a sleeve for the arm, and utilizes a removable pad that is inserted into the palm area rather than affixed to the outer surface. The bulky padding can interfere with athletic activities such as ball handling and does not address the needs of athletes seeking effective sweat control.

U.S. Pat. No. 7,690,050 B 2 discloses an armband featuring a receptacle designed to hold multiple disposable absorbent pads for capturing byproducts expelled from the mouth and nose. This device is specifically intended for hygiene purposes rather than for managing perspiration, and the absorbent strip is positioned within the receptacle, rendering the design inappropriate for wiping sweat from the body. The configuration does not support the mechanical transfer of perspiration from the skin to an external absorbent surface.

U.S. Patent Application Publication No. 2002/0213866 A1 describes articles of clothing with water-absorbent portions, such as a removable absorbent strip on the forearm, shoulder, or midsection. While this approach provides enhanced sweat absorption compared to a compression sleeve alone, the design depends entirely on absorbent material, which can become wet, heavy, and uncomfortable during activity. Placement of absorbent strips on sensitive areas like the anterior forearm or shoulder can also disrupt balance, obstruct vision, and compromise running posture, resulting in reduced athletic performance and comfort.

With pickleball injuries up 700% from 2013-2022 and hospital admissions up 257% during this same period, Floky™ (floky.us) designed a “No Strain Sleeve” bio-mechanical compression technology that reportedly decreases muscle vibrations during the rapid directional changes, explosive arm movements, and repetitive swinging motions that particularly stress the forearms and elbows of the world's 36.5 to 48 million current pickleball players during extended play sessions. However, Floky's sleeve does not combine sleeve compression with a dual system of sweat management nor does it provide proof or a statement of greater, and thus patentable, vibration reduction above standard compression sleeves on the market. Additionally, an EICEM Pickleball sleeve (https://www.amazon.sa/-/en/EICEM-Pickleball-Protection-Comfortable-Accessories/dp/B0CR14HJHS) suffers from similar limitations as the Floky sleeve.

Despite these developments, there remains a significant gap in the market for a garment that provides both the benefits of compression and effective sweat collection without compromising performance. Existing solutions are often bulky, uncomfortable, or poorly positioned for optimal sweat management during athletic activity.

The anterior forearm is more touch-sensitive than dorsal or lateral regions, especially distally, due to higher receptor density and smaller receptive fields (Purves D, Augustine G J, Fitzpatrick D, et al., eds. Neuroscience. 2nd ed. Sinauer Associates; 2001. Differences in Mechanosensory Discrimination Across the Body Surface. Available at: https://www.ncbi.nlm.nih.gov/books/NBK11088/#; Mancini F, Bauleo A, Cole J, Lui F, Porro C A, Haggard P, Iannetti G D. Whole-body mapping of spatial acuity for pain and touch. Ann Neurol. 2014; 75(6):917-24. doi:10.1002/ana.24179. PMID: 24816757; PMCID: PMC4143958). Placing a water-absorbent strip there, which becomes wet and heavy, increases discomfort and distraction. Locating sweat-management surfaces on less sensitive regions and using moisture-wicking or dual-layer constructions that stay drier mitigates irritation and better supports athletic performance.

Forearm-and shoulder-placed absorbent strips can degrade safety and performance. To use a forearm strip, athletes must lift and sweep the arm across the face during motion, disrupting arm-leg counterbalance, increasing torso rotation, reducing stability, and raising energy cost, while also obstructing vision and breaking concentration. Shoulder placement prompts forward head tilt to wipe, which disrupts vestibular inputs, removes the eyes from the horizon, and degrades running posture and efficiency, increasing effort and instability (Wenger P C, MD. Straightening the Stride: The Impact of Poor Posture on Running Performance. Feb. 1, 2024. https://www.princetonmedicine.com/blog/straightening-the-stride-the-impact-of-poor-posture-on-running-performance). Consequently, such placements are prone to discomfort, visual impairment, balance disturbance, and heightened risk, making them less safe and useful than athlete-centric configurations.

Still another problem that athletes face is soles of their shoes becoming filled with dust or other debris, which functions to limit the effectiveness of the sole gripping the ground. For example, if an athlete makes a quick cut or stop, if the sole of the shoe has debris located therein, there is a significant increase in the risk of slipping thereby increasing injury risk. While sneaker brushes and traction mats are commonly used in between game play or during breaks, these require the athlete to leave the court. None of the above-discussed articles disclose or teach an article that allows the athlete to remove debris from their shoes without having to leave the court. Accordingly, there is a pronounced need for a streamlined athletic garment that combines the performance and recovery benefits of compression with an integrated yet detachable system for both moisture-wicking and high-capacity sweat absorption. Such a solution is designed to maintain secure placement throughout rigorous activity, provide rapid access to sweat-collecting surfaces without interrupting play, and avoid the bulk and inconvenience associated with existing sleeve attachments or standalone accessories. A carefully configured arrangement of sweat-handling elements, optimally positioned and easily replaceable, would fulfill the requirements of modern athletic performance.

### SUMMARY

Accordingly, what is needed is an article for managing perspiration and providing compression therapy during physical activity.

An article is further desired to be provided, comprising a compression sleeve with a proximal opening designed to accommodate a limb and a distal opening designed to accommodate a hand.

A further objective is to provide at least one detachable sweat-management strip secured to an exterior surface of the compression sleeve, the strip being formed of moisture-wicking and/or absorbent material and designed for removal, replacement, or repositioning at one or more locations along the sleeve during use.

It is further desired to provide a combination compression sleeve that features a pickleball/tennis ball sheath that can be used throughout a game to conveniently and more ergonomically house several spare pickleballs on its surface that can be accessed by a contralateral hand, thereby preventing athletes from having to chase balls around the court during a game.

It is also desired to provide a combination compression arm sleeve with an integrated cleaning surface(s) that can be used throughout the game to conveniently remove debris from the sole of a shoe, thereby preventing athletes from having to leave the court and interrupt their play.

The strip is also desired to combine both moisture-wicking and absorbent layers and to attach via hook-and-loop fasteners.

The strip may be positioned in regions such as the dorsal radial area, anterior forearm, wrist, bicep, or palm-knuckle.

Additionally, there is a desire to provide that the compression sleeve incorporates a fingerless glove portion at the distal end, where the fingerless glove portion features a thumb hole, multiple finger holes, and a release slit for convenient removal.

The present design addresses these limitations by introducing a compression sleeve integrated with detachable dual moisture-wicking and absorbent sweat strips. This approach combines the performance benefits of compression therapy with advanced sweat management capabilities, ensuring athletes remain dry, comfortable, and focused during physical activity. The detachable sweat strips, secured via hook-and-loop fasteners, are strategically positioned along the sleeve to optimize accessibility and functionality. By incorporating both moisture-wicking and absorbent layers, the strips effectively manage perspiration while maintaining lightweight comfort. The compression sleeve further includes features such as a fingerless glove portion with thumb and finger holes for enhanced stability, an anti-slip structure to prevent downward migration, and stretchable materials like spandex or nylon for flexibility and durability. These elements collectively provide a streamlined, high-performance solution that overcomes the bulk, discomfort, and inefficiency of existing designs, enabling athletes to manage sweat effectively without compromising their mobility or focus.

Positioning a sweat strip along the dorsal-radial bony plane reduces visual obstruction during forehead wiping compared to lower forearm placements, improving both safety and utility for athletes. Additionally, when a humeral strip is oriented perpendicularly along the side of the face and used in concert with a horizontally oriented dorsal-radial strip across the forehead, the two form a V-shaped wiping interface. This configuration enables a quick “V-pat” motion that clears sweat with minimal arm elevation, preserves the visual field, and maintains balance better than anterior forearm strip locations.

In some embodiments, a proximal anti-slip structure is desired to be provided, which is disposed to engage the shoulder or head to prevent downward migration of the sleeve.

In one configuration, constructing the sleeve from stretchable materials such as spandex or nylon is desired.

In another configuration, a plurality of independently detachable sweat-management strips is desired, which can be wrung out without requiring full detachment from the sleeve.

In another embodiment, a method is desired for managing perspiration and providing compression therapy during physical activity.

In one embodiment, the compression sleeve includes a placement on the anterior forearm over the flexor-pronator muscle group for a moisture-wicking strip configured to remove perspiration from the face, head, neck, or body while maintaining a drier, more comfortable interface on this highly sensitive region. Unlike water-absorbent strips that retain fluid and become heavy and uncomfortable during gameplay, the moisture-wicking strip promotes rapid capillary transport and evaporation. The strip may extend substantially along the longitudinal length of the anterior forearm or be provided as multiple inserts that extend at least partially around the sleeve, and may be dimensioned in length, width, and thickness to surpass the sweat-handling capacity of typical wristbands and headbands. In all cases, the strip is an open-faced element removably affixed to the exterior of the sleeve to permit replacement, repositioning, or removal during use.

The compression sleeve includes a placement at the wrist for a moisture-wicking strip configured to maintain a drier, more comfortable interface over the sensitive anterior wrist region during and after wiping. The strip may extend circumferentially around at least a portion of the wrist and is dimensioned in length, width, and thickness to handle low to moderate perspiration from the face, head, neck, and body, with sweat-handling capacity exceeding that of typical wristbands and headbands. The strip is provided as an open-faced element removably affixed to the exterior of the sleeve to permit replacement, repositioning, or removal during use. Technical effect: rapid capillary transport and evaporation reduce retained moisture and associated discomfort compared to water-absorbent strips that become heavy and wet during gameplay.

Accordingly, the method comprises: providing the compression sleeve with a proximal opening for a limb and a distal opening for a hand; securing at least one detachable sweat-management strip to an exterior surface of the sleeve; and removably attaching the strip at one or more locations along the sleeve to facilitate removal, replacement, or repositioning during use.

The method is further desired to include using strips that combine moisture-wicking and absorbent materials, with the strips being secured via hook-and-loop fasteners.

The method is still further desired to include positioning the strips at selected regions of the sleeve, incorporating a fingerless glove portion with a release slit, employing an anti-slip structure to prevent migration, constructing the sleeve from stretchable materials, and providing multiple independently detachable strips that can be wrung out without removal.

For this application the following terms and definitions shall apply:
The terms “first” and “second” are used to distinguish one element, set, data, object or thing from another, and are not used to designate relative position or arrangement in time.
As used herein, the phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.
The terms “coupled”, “coupled to”, “coupled with”, “connected”, “connected to”, and “connected with” as used herein each mean a relationship between or among two or more devices, apparatus, components, systems, subsystems, and/or means, constituting any one or more of (a) a connection, whether direct or through one or more other devices, apparatus, components, systems, subsystems, or means, (b) a communications relationship, whether direct or through one or more other devices, apparatus, components, systems, subsystems, or means, and/or (c) a functional relationship in which the operation of any one or more devices, apparatus, components, systems, subsystems, or means depends, in whole or in part, on the operation of any one or more others thereof.

In one embodiment an article for managing perspiration and providing compression therapy during physical activity is provided comprising a compression sleeve having a proximal end with an opening for receiving a limb and a distal end with an opening for receiving a hand. The article further comprises least one detachable sweat-management strip secured to an exterior surface of the compression sleeve, the at least one sweat-management strip comprising at least one of a moisture-wicking material and an absorbent material, wherein the at least one sweat-management strip is configured to be removably attached at one or more locations along the compression sleeve to facilitate removal, replacement, or repositioning of the sweat-management strip during use. The article is provided such that the compression sleeve and the at least one sweat-management strip are adapted to collectively provide both compression and enhanced perspiration management for a user during physical activity.

The above-described and other features and advantages of the present disclosure will be appreciated and understood by those skilled in the art from the following detailed description and accompanying drawings.

### DESCRIPTION OF THE DRAWINGS

FIG. 1 is an illustration of the combination compression sleeve and absorbent material showing the outside edge (facing away from the body) of the article according to a configuration of the invention.

FIG. 2 is an illustration of the combination compression sleeve and absorbent material, showing the inside edge (facing toward the body) of the article, according to FIG. 1.

FIG. 3 is an illustration of the absorbent material disconnected from the compression sleeve according to a first configuration based on FIG. 2.

FIG. 4 is an illustration of the absorbent material disconnected from the compression sleeve according to a second configuration based on FIG. 2;

FIG. 5 is an illustration of the absorbent material connected to the compression sleeve according to FIG. 4;

FIG. 6 is a cross-sectional diagram along Section A-A according to FIG. 5;

FIG. 7 is an illustration of the absorbent material disconnected from the compression sleeve according to FIG. 3 and further includes an absorbent material extending around a wrist portion of the compression sleeve;

FIG. 8 is an illustration of the absorbent material with a back bar and additional shoulder structure according to the configuration of FIG. 1; and

FIG. 9 is an illustration of the absorbent material with an additional compression sleeve according to the configuration of FIG. 8.

FIG. 10 is a perspective view of the compression sleeve with a distal end terminating in a fingerless glove portion with a thumb hole, finger holes, and a release slit, and illustrating exemplary detachable strips positioned at the dorsal radial region, anterior forearm region, wrist region, bicep region, and palm-knuckle region according to the configuration of FIG. 1.

FIG. 11 is a perspective view of the fingerless glove portion according to the configuration of FIG. 10.

FIG. 12 is a perspective view of the compression sleeve with a neck loop according to the configuration of FIG. 10.

FIG. 13 is a side view of the compression sleeve with a sheath affixed to an exterior surface thereof according to FIG. 1.

FIG. 14 is a side view of the compression sleeve according to FIG. 13 further including a cleaning surface with debris-removal structures.

### DETAILED DESCRIPTION

The following detailed description provides illustrative embodiments of the described technology, which pertains to the field of athletic wear and sweat management systems. Specifically, the described technology integrates a compression sleeve with detachable dual moisture-wicking and absorbent sweat strips to enhance comfort, performance, and perspiration control during physical activity. While the embodiments described herein focus on particular configurations and features, it is important to understand that these examples are provided for illustrative purposes only and are not intended to limit the scope of the described technology. The described technology may be adapted, modified, or rearranged in various ways without departing from its intended purpose or scope.

Certain well-known principles, components, and techniques commonly understood by those skilled in the art may not be described in detail herein to avoid redundancy and maintain clarity. The described subject matter includes numerous variations, such as alternative materials, attachment mechanisms, and sweat strip placements, all of which fall within the scope of the claims. Accordingly, the detailed description is to be read with the understanding that the described subject matter is flexible and adaptable to meet diverse user needs and applications.

The present disclosure relates to an article for managing perspiration and providing compression therapy during physical activity. The article integrates a compression sleeve with at least one detachable sweat-management strip formed of moisture-wicking and/or absorbent materials. The sweat-management strip is configured to be removably attached at one or more locations along an exterior surface of the compression sleeve to facilitate removal, replacement, repositioning, or wringing out of moisture during use, while the compression sleeve and the sweat-management strip collectively provide both compression and enhanced perspiration management.

In the embodiments described herein, reference is made to figures illustrating preferred constructions, placements, and configurations of the compression sleeve and detachable sweat-management strips. The examples are provided for clarity in understanding the scope of the invention and are not limiting; variations in materials, geometries, and attachment layouts may be made without departing from the spirit or scope of the claims.

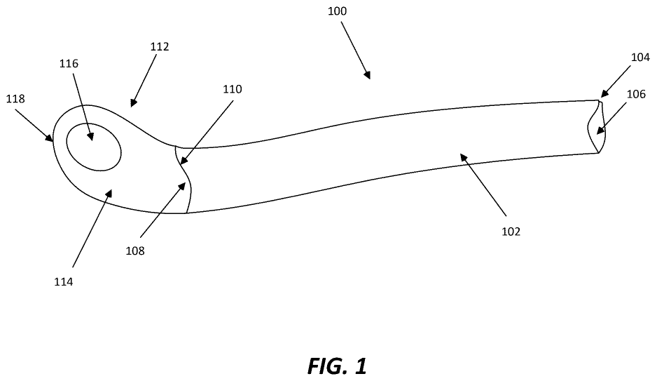

Referring to the drawings and in particular, to FIG. 1, an article 100 is provided that comprises a compression sleeve 102 including a distal end 104 with a distal opening 106, and a proximal end 108 with a proximal opening 110. It is contemplated that the compression sleeve may comprise a relatively thin, nylon and spandex material and applies an even compression to the limb within the sleeve.

The article 100 further comprises an anti-slip structure 112 that comprises an elongated material 114 extending from the proximal end 108. The elongated material further includes an opening 116 positioned therein to allow a user to insert their arm through opening 116.

In function, a user would pass their hand and arm through the opening 116 and then into proximal opening 110 and finally out through distal opening 106. When article 100 is fully seated on the arm, the upper end 118 elongated material 114 will sit over the top of the user's shoulder with the elongated material 114 running along an inner side of the user's arm. This anti-slip structure 112 can be made of the same material as the compression sleeve 102 and can be formed ergonomically to comfortably conform to the user's shoulder. In this manner the compression sleeve will be prevented from slipping or traveling down the user's arm as the upper end 118 prevents any downward longitudinal movement.

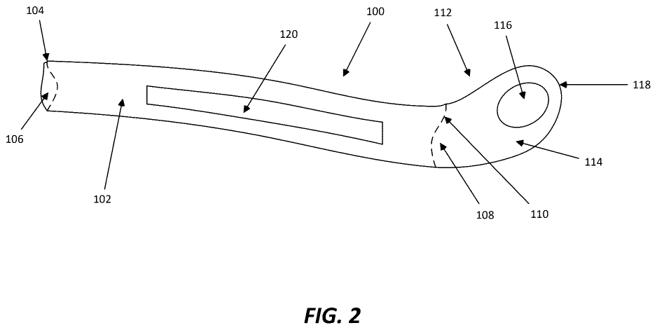

FIG. 2 is a view of article 100 looking at the inside portion (facing toward the body) of the compression sleeve 102 and showing the absorbent material 120. Based on the configuration, a user would put their arm through the opening 116 and then into the proximal opening 110 with the user's hand extending out of distal opening 106. In this configuration, the absorbent material 120 faces toward the user making it easy to use the absorbent material 120 to wipe away sweat from the user's head, face, neck and body. It is contemplated that the absorbent material 120 may comprise a fabric or cloth material.

The absorbent material 120 is situated or positioned along the inner seam of the compression sleeve 102, however, it is contemplated that the compression sleeve 102 could be rotated axially around the arm to radially position the absorbent material 120 on the arm as desired (e.g., the compression sleeve 102 could be rotated such that the absorbent material 120 is radially positioned towards the inner top of the arm) being limited only by the slip structure 112 and upper end 118 that loops over the user's shoulder. It should be noted that the absorbent material could run along a longitudinal length of the compression sleeve or may be formed as a absorbent material extending at least partially radially around the compression sleeve. Additionally, rather than only one absorbent material, the compression sleeve could be adapted to function with multiple discrete absorbent materials that are coupled to the compression sleeve. For example, a relatively short absorbent material could be provided on the inside of the wrist and a second longitudinally extending absorbent material could be provided on the inside edge of the forearm. While the figures illustrate one configuration, the invention is not limited to only a longitudinally extending absorbent material.

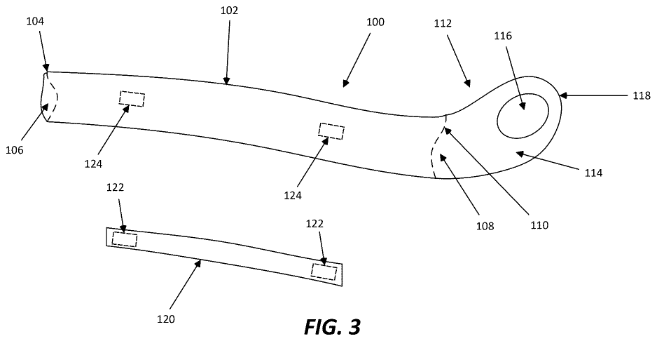

FIG. 3 illustrates a first embodiment of the invention according to the configurations of FIGS. 1 and 2. Article 100 is shown with the absorbent material 120 disconnected from the compression sleeve 102. Also illustrated is a securing mechanism that comprises a hook fastener 122 that is adapted to engage with a loop fastener 124. While only two sets of hook 122 and loop 124 fasteners are shown, it is contemplated that any number of fasteners may be positioned along a longitudinal length of the absorbent material 120. In another configuration, a hook 122 and loop 124 fastener may extend along a complete longitudinal length (or any portion thereof) of the absorbent material 120 to ensure it is firmly held to the compression sleeve 102.

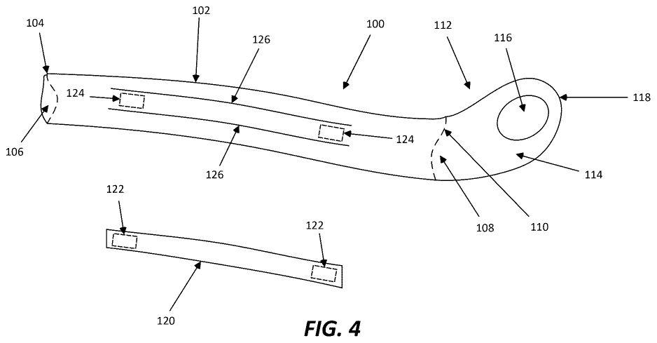

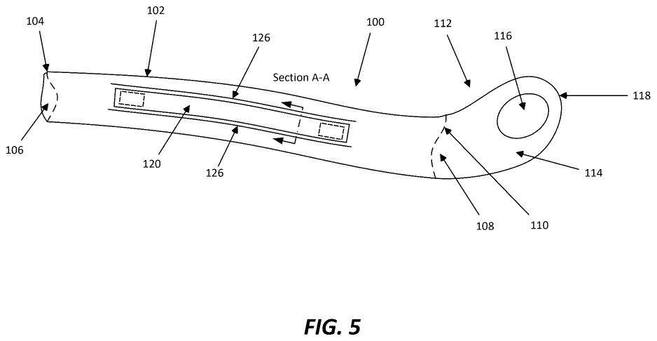

FIGS. 4 and 5 illustrate a second embodiment of the invention according to the configurations of FIGS. 1 and 2. Article 100 is again shown with the absorbent material 120 disconnected from the compression sleeve 102 (FIG. 4). In this configuration, track 126 is shown on the surface of compression sleeve 102 into which the absorbent material 120 is adapted to be positioned. The track 126 may be affixed (e.g., sewn) into the compression sleeve 102. In one configuration, track 126 may comprise a plastic material that is semi-rigid but allows for flexibility to conform to the curvature of the athlete's arm.

FIG. 5 shows the absorbent material 120 inserted into track 126 and held in place via a securing mechanism, which may, in one configuration, comprise a hook and loop fastener.

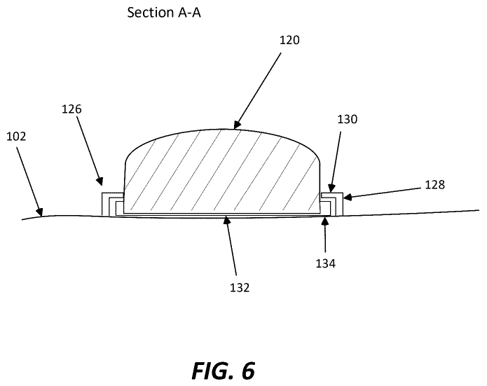

Referring now to FIG. 6, a cross-section of Section A-A (FIG. 5) is illustrated. The track 126 is shown comprising an upstanding wall 128 and formed with an undercut 130.

The absorbent material is further shown having a backing 132, that includes ends 134 that are adapted to engage with the undercut 130. The drawings are not shown to scale, and it is contemplated that the ends 134 may comprise a very low profile structure or tab (e.g., the backing may or may not extend upward around the side of the absorbent material 120). It is contemplated that the absorbent material 120 may be affixed to the backing by an adhesive.

Additionally, it is contemplated that the backing 132 may comprise a semi-rigid material that is deformable allowing the absorbent material 120 to easily be inserted and removed from the track.

In one configuration, backing 132 is provided with a securing mechanism as previously described. In other configuration, the backing 132 may be sized to fit tightly within track 126 such that the absorbent material 120 is held in place via a friction fit between the ends 134 and the undercut. To insert or remove the absorbent material 120, a user simply has to grab the absorbent material 120 at one end and pull upward and the backing 132 will deform allowing removal. Alternatively, the user could simply slide the absorbent material 120 out of an end of the track 126. In any event, a quick and easy change of the absorbent material 120 is possible in just a few seconds if the athlete needs to replace the absorbent material 120 during a game (e.g., during a time out).

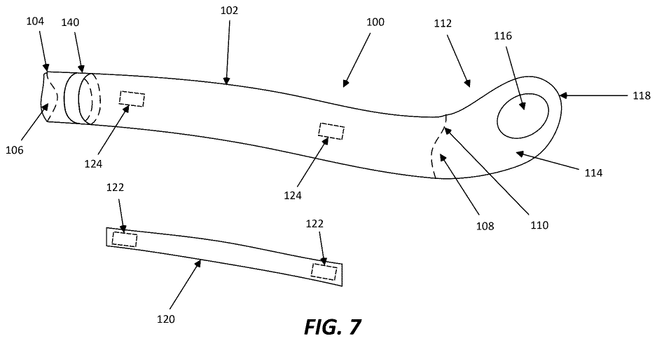

FIG. 7 is a configuration similar to FIG. 3 but further includes an absorbent material 140 positioned around a wrist portion of the compression sleeve 102. In this configuration, it is contemplated that the absorbent material 140 may be removable from the compression sleeve 102 and may further include a securing mechanism as previously discussed. In one configuration, the absorbent material 140 may extend around a radial portion of the compression sleeve 102 (e.g., 120 degrees, or 180 degrees). In this configuration, the securing mechanism may include, for example, the hook and loop fastener or the track or both. In another configuration, the absorbent material 140 may extend completely around compression sleeve 102. In this configuration, the absorbent material 140 may comprise a stretchable material that may be used with the securing mechanism. The absorbent material 140 can also serve to absorb the shock/force of a basketball rim exerted on an athlete's dorsal wrist during dunks or other types of physical contact.

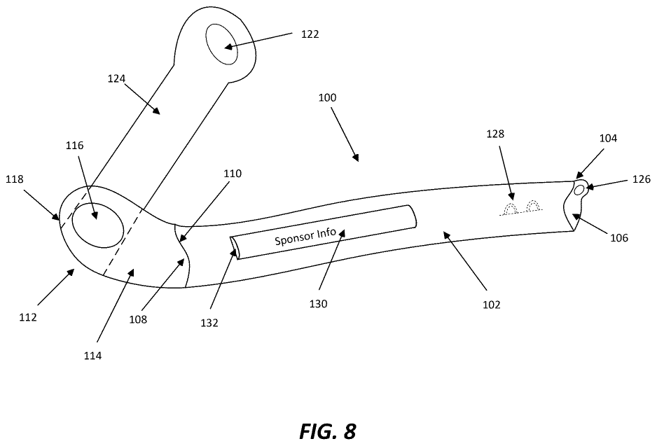

Referring now to FIG. 8, an alternate configuration to article 100 shown in FIG. 1 is provided. In this configuration, a back bar 124 is connected to upper end 118 and terminates with a shoulder structure 136 having an opening 122 positioned therein.

In function, a user would pass their left arm and hand through opening 122 so that it is fully seated over the top of the user's left shoulder; where opening 122 is also attached along its rear seam to a back bar 124 comprised of an elongated piece of material, (that could also be thick and absorbent). This configuration will function to more securely hold the compression sleeve in place preventing any type of downward movement.

In another configuration, the compression sleeve also features a thumb hole 126 for securing and/or stabilizing the compression sleeve on the arm during use.

In still another configuration, the compression sleeve also features 128 silicone or rubberized stress pulls or fidgets to help athletes release stress and anxiety during breaks or while seated on the bench.

In yet another configuration, the compression sleeve also features a clear slot 130 for inserting through an opening 132 various placards or pictures displaying athletes' NIL deals or other types of advertising.

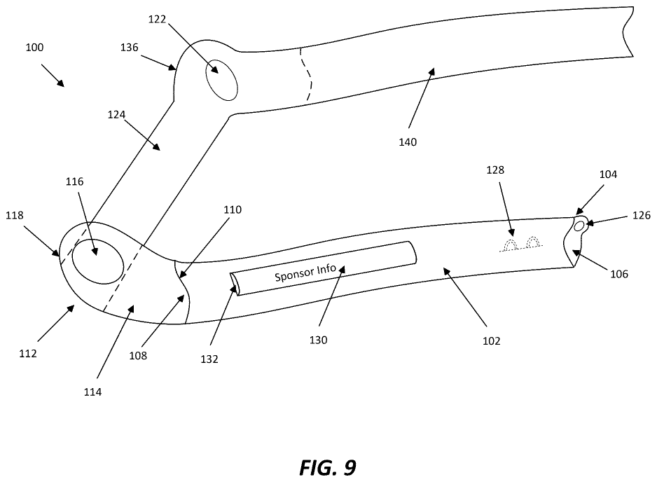

Turning now to FIG. 9 still another configuration is shown. In this embodiment, a second arm 140 is shown connected to the shoulder structure 136. The second arm 140 may include some or all the various features discussed in connection with the previous FIGS and will not be redescribed here. Likewise, the back bar 124 may include a removable sweat insert as discussed in connection with the compression sleeves or may simply comprise the same material forming the compression sleeves.

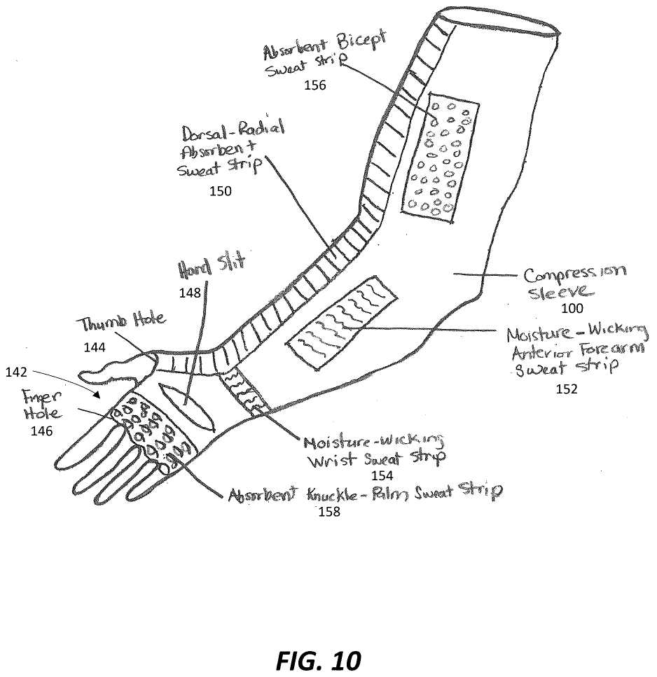

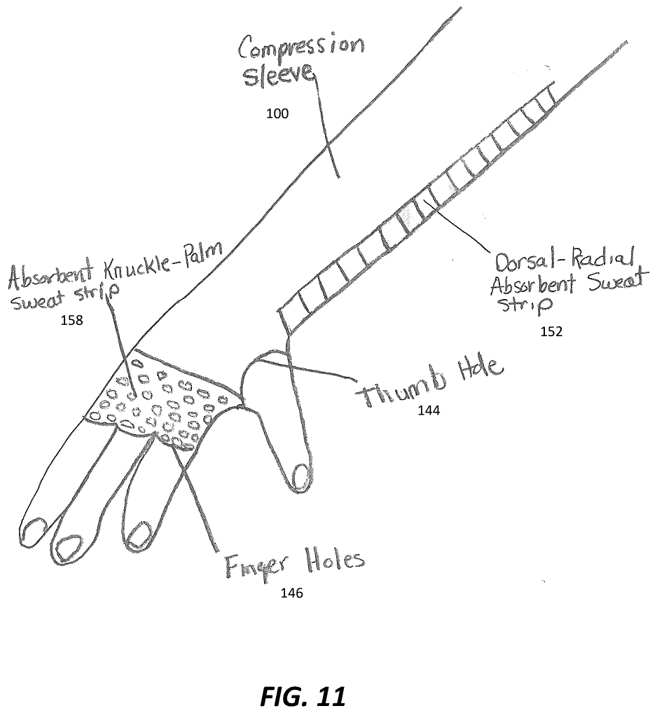

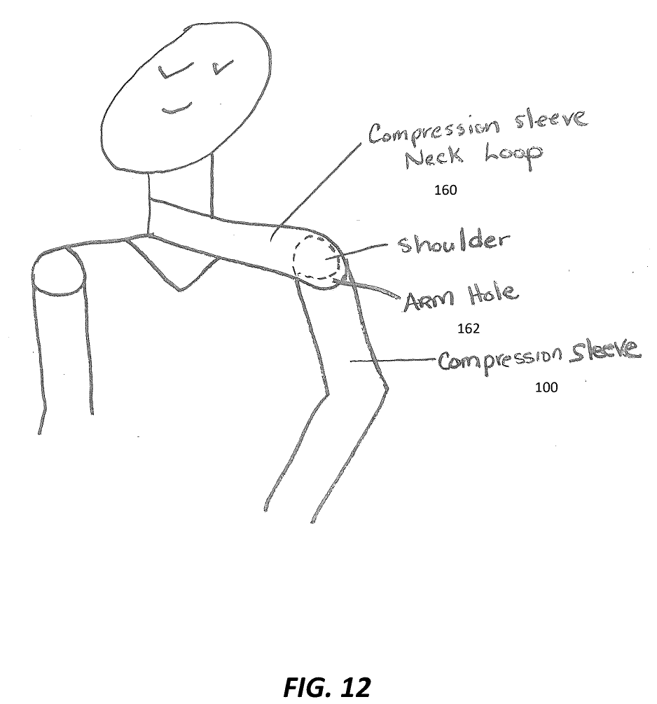

Referring now to FIGS. 10-12, another example of the compression sleeve 100 is provided that includes a distal end that culminates in a fingerless glove portion 142 having a thumb hole 144 and a plurality of finger holes 146. A release slit 148 may be provided in the fingerless glove portion 142 to allow convenient removal of the hand without removing the sleeve from the limb.

At least one detachable sweat-management strip is secured to an exterior surface of the compression sleeve 100. In certain embodiments, each sweat-management strip comprises a dual-layer construction including a moisture-wicking layer disposed outwardly to promote capillary transport and evaporation, and an absorbent layer disposed inwardly to increase storage capacity and to buffer transient surges in perspiration. In other configurations, strips may be formed of only moisture-wicking material or only absorbent material, and combinations of strips may be deployed concurrently to tailor performance for individual athletes and use cases. Attachment is achieved by any acceptable method including, for example, complementary hook-and-loop applied to the sleeve and the underside of each sweat-management strip. Fastener fields may extend substantially along the length and/or width of the strip to promote uniform engagement, reduce edge lift during wiping, and allow secure repositioning.

Preferred placement regions include a dorsal radial region strip 150 along the posterior-lateral plane of the forearm, an anterior forearm region strip 152 positioned along the flexor-pronator muscle group, a wrist region strip 154, a bicep region strip 156, and a palm-knuckle region strip 158 mounted on the fingerless glove portion 142, spanning laterally across the metacarpophalangeal area. Strategic placements improve reachability, minimize visual obstruction, and maintain the athlete's biomechanical rhythm during running and gameplay. A plurality of independently detachable strips may be provided and deployed simultaneously on the compressive sleeve 100. Each strip may be wrung out by hand without detaching from the sleeve, allowing on-the-spot moisture removal and rapid restoration of usable absorbency during play.

To prevent downward migration of the compression sleeve 100 along the limb during vigorous activity, an anti-slip proximal structure 160 may extend from the proximal end of the sleeve. The anti-slip structure 160 defines an upper opening 162 sized to receive and sit over the user's shoulder, thereby redistributing load paths and stabilizing the sleeve during repeated wiping actions that impart downward forces. In alternative embodiments, an anti-slip head/neck variant may be provided, wherein the structure extends over the neck and/or head to resist sleeve migration. The anti-slip structures 160 may be elastic and may incorporate knitted or laminated features for comfort and fit.

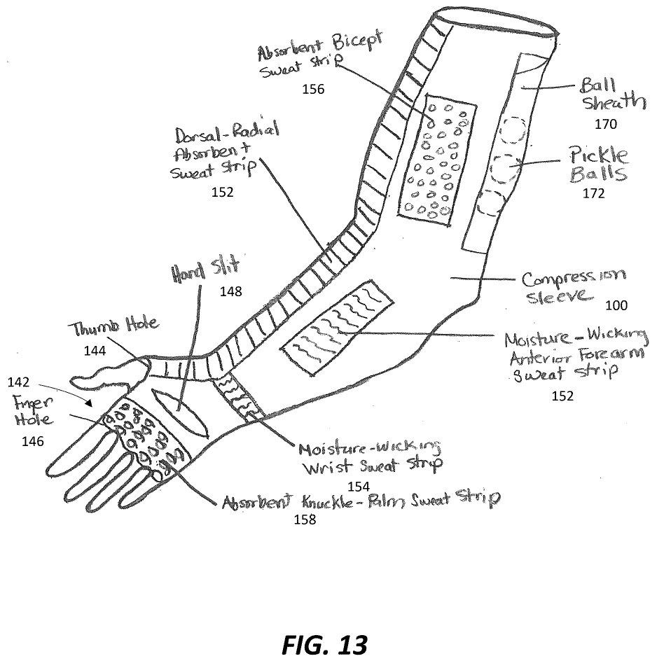

Referring to FIG. 13, another configuration of the compression sleeve 100 is illustrated that includes a sheath-like structure 170 on the outer surface of the compression sleeve 100 extending the length of the tricep area and beyond with enough length to tightly house and conceal at least two pickleball/tennis balls 172 upon insertion into the distally-located sheath opening, allowing athletes to remain in place on the court and simply pull out another ball to use when a prior ball has gone out of bounds, bounced off, or rolled away from them, thereby conserving their energy for actual play.

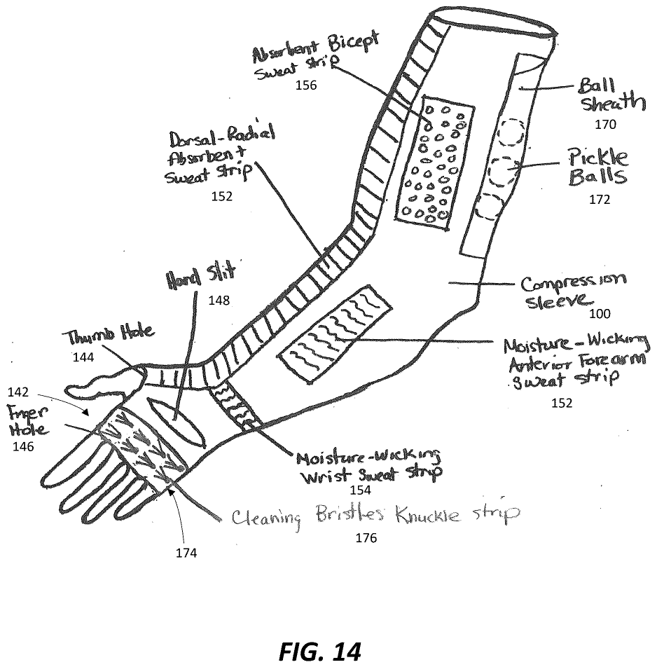

FIG. 14 illustrates the compression sleeve of FIG. 13, however, it is provided with a strip 174 of structural configurations 176 (e.g., stiff bristles) including: 1) flexible rubber spikes or nubs; 2) silicone spike patch; 3) serrated scraping bars with tooth-like edges; 4) hook-and-loop structures (i.e., Velcro®); 5) hard grid or lattice structure; or 6) an abrasive strip of material, positioned near the distal end of the sleeve, which is removably affixed to and stabilized by the main knuckles of the hand. It is contemplated that bending motion of the hand facilitates the various structural configurations 176 penetrating tread grooves to loosen/remove debris. The strip 174 extends longitudinally, from the mid-palm to the main knuckles of the hand and, horizontally, from the index finger to the pinky, and longitudinally across the wrist, which can also be radially affixed. The structural configurations 176 offer athletes particular advantages and greater utility over prior art articles/devices as it allows athletes to remove debris from the soles of their shoes while mobile and remaining in play on the court and is integrated with arm compression and sweat management article as opposed to having to use a brush or a mat.

This configuration offers particular advantages and greater utility over existing compression sleeves including: bio-mechanically superior inertial positioning; lower interference with both arm swing andc pronation mechanics; less visual field and kinesthetic obstruction than lateral arm and forearm placements; an easy reach for the contralateral hand; improved dynamic compression stability compared to forearm, lateral arm, waistband, or loose pocket placements; as well as, minimal interference with high-frequency forearm movements, making it both more ergonomic, useful, and safe for athletes than the prior art.

It is contemplated that the ball retaining sheath can essentially run the entire length of the posterior upper arm (tricep) allowing the athlete to store and access at will several small playing balls than existing compression sleeves provided by the prior art. In this configuration, the ball-retaining sheath is essentially an open-faced strip of material affixed to the outer surface of the compression sleeve. Alternatively, the sheath could comprise multiple individual sheaths affixed to the outer surface of the compression sleeve.

In one configuration, an article comprising a compression sleeve with a proximal opening and distal opening and at least one detachable sweat-management strip secured to an exterior surface by fastener fields. A single strip comprising moisture-wicking material is mounted at the dorsal radial region. In another embodiment, a single absorbent strip is mounted at the bicep region. In yet another embodiment, a dual-layer strip is mounted at the anterior forearm region. Technical effect: the strip(s) enhance sweat capture and evaporation while the sleeve provides compression therapy, reducing muscle oscillation and supporting circulation; the modularity allows users to adapt sweat-handling capacity and wiping ergonomics mid-activity without removing the sleeve.

In another configuration, the strip comprises both moisture-wicking material and absorbent material. The moisture-wicking layer may be outward-facing to maximize capillary transport, while the inward absorbent layer buffers transient perspiration peaks. Technical effect: the dual-layer arrangement improves evaporation kinetics and storage capacity, preventing over-saturation, reducing dripping, and maintaining a drier contact surface for repeated wiping events.

In still another configuration the strip is secured by hook-and-loop fasteners. The sleeve integrates continuous hook fields at regions, and each strip incorporates complementary loop fields on its underside. Technical effect: rapid attachment, removal, and repositioning are enabled with robust shear resistance during wiping, reducing strip displacement and improving reliability in competitive play.

In another configuration the strip is positioned at a location selected from the dorsal radial region, anterior forearm region, wrist region, bicep region, and palm-knuckle region. Two strips are used concurrently: a dorsal radial strip for forehead wiping and a palm-knuckle strip for periocular wiping, both reachable with minimal arm diversion from natural running counterbalance. Technical effect: optimized placement reduces visual obstruction, preserves running posture, and minimizes energy expenditure associated with large arm movements, improving safety and performance.

In yet another configuration the compression sleeve includes a fingerless glove portion with thumb hole and finger holes. In an embodiment, glove portion stabilizes the palm-knuckle strip against knuckle contours, improving precise wiping of high-sensitivity areas without bulk in the palm. Technical effect: increased control during wiping and reduced interference with ball handling or object manipulation.

In still another configuration the fingerless glove portion includes a release slit 112. A slit allows the hand to be extracted while the sleeve remains on the forearm; the palm-knuckle strip can be folded inward when not in use. Technical effect: rapid convertibility between gloved and non-gloved states enhances usability for sports requiring bare-hand grip while preserving sweat-management capacity on the forearm.

In one configuration the article includes an anti-slip structure extending from the proximal end defining an upper opening to sit over the shoulder; in an alternative variant, the structure sits over the head/neck. Technical effect: the anti-slip structure redistributes forces from wiping actions to proximal anatomical anchors, preventing downward migration of the sleeve, maintaining strip alignment, and reducing distraction and re-adjustment frequency.

In another configuration the strip can be wrung out without detaching. The dual-layer strip is flexible and compressible, and the fastener field has sufficient shear strength to remain engaged while fluid is expelled by squeezing. Technical effect: on-the-spot restoration of absorbency extends on-field usability, reduces downtime, and minimizes the need for replacement during a game.

In still another configuration the sleeve is constructed of stretchable materials such as spandex, nylon, or combinations thereof. The sleeve features zoned elastic recovery tailored to muscle groups along the forearm and bicep. Technical effect: compression therapy reduces muscle oscillation, may support venous return, and improves proprioception, while the smooth textile base provides a consistent substrate for strip engagement.

In yet another configuration the strip(s) are provided as a plurality of independently detachable and replaceable strips. A kit is provided with several strips of varied lengths and material combinations (moisture-wicking material or absorbent material or both). Technical effect: modularity enables personalized sweat-handling strategies, quick mid-activity reconfiguration, and efficient hygiene management by swapping soiled strips without removing the sleeve.

The compression sleeve may be formed by circular knitting or warp knitting processes using spandex and nylon yarns. The fingerless glove portion may be integrally knitted with reinforced apertures for thumb hole and finger holes, and a bonded or stitched release slit. The fastener fields may be sewn or bonded to the sleeve exterior at regions. Each sweat-management strip may be fabricated as a laminate with an outward moisture-wicking layer (e.g., hydrophilic polyester microfiber or capillary knit) and an inward absorbent layer (e.g., terry or high-loft absorbent fabric), with a backing incorporating complementary hook-or-loop tape for engagement. Edges may be bound to resist fraying and to improve durability under repeated wringing. Anti-slip structures may be cut-and-sewn or knitted extensions with elasticized rims forming upper openings sized to sit over the shoulder or head/neck, respectively, and may incorporate comfort linings and moisture management panels.

In operation, the user puts on the compression sleeve by inserting the limb through proximal opening. When present, the fingerless glove portion stabilizes at the hand via thumb hole and finger holes, while the release slit allows rapid conversion to a bare-hand configuration. The user applies one or more sweat-management strips onto fastener fields at selected locations depending on anticipated perspiration and wiping preferences. During activity, the user wipes the face, head, neck, or body onto outward-facing moisture-wicking surfaces to promote rapid capillary transport and evaporation while absorbent layer buffers moisture surges. When a strip accumulates excessive moisture, the user wrings the strip in situ to expel fluid and continue activity without removing the sleeve. Alternatively, replacement strips could be quickly applied. The anti-slip structures prevents sleeve migration during repeated wiping strokes, maintaining consistent strip alignment and improving overall usability.

The described article unifies compression therapy with a detachable, high-performance sweat-management system. The dual-layer strip architecture, modular placements, and in-situ wringing/replacement capability collectively mitigate saturation, dripping, and distraction, while hook-and-loop fasteners enable rapid replacement and repositioning. The fingerless glove portion with release slit improves wiping precision and convertibility for ball-handling sports. Proximal anti-slip structures stabilize the sleeve under dynamic wiping loads. Construction from spandex/nylon blends provides requisite stretch, durability, and base-surface compatibility with fasteners. Overall, the configuration enhances athlete comfort, safety, and performance by minimizing visual obstruction, preserving biomechanical rhythm, and optimizing sweat-handling capacity throughout rigorous activity.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

What is claimed is:

1. An article for managing perspiration and providing compression therapy during physical activity, comprising:
a compression sleeve having a proximal end with an opening for receiving a limb and a distal end with an opening for receiving a hand;
least one detachable sweat-management strip secured to an exterior surface of the compression sleeve, the at least one sweat-management strip comprising at least one of a moisture-wicking material and an absorbent material, wherein the at least one sweat-management strip is configured to be removably attached at one or more locations along the compression sleeve to facilitate removal, replacement, or repositioning of the sweat-management strip during use;
wherein the compression sleeve and the at least one sweat-management strip are adapted to collectively provide both compression and enhanced perspiration management for a user during physical activity.

2. The article of claim 1, wherein the at least one sweat-management strip comprises both a moisture-wicking material and an absorbent material.

3. The article of claim 1, wherein the at least one sweat-management strip is secured to the compression sleeve by a hook-and-loop fastener.

4. The article of claim 1, wherein the at least one sweat-management strip is positioned at a location selected from the group consisting of a dorsal radial region, an anterior forearm region, a wrist region, a bicep region, and a palm-knuckle region of the compression sleeve.

5. The article of claim 1, wherein the compression sleeve further comprises a fingerless glove portion at the distal end, the fingerless glove portion including a thumb hole and a plurality of finger holes.

6. The article of claim 5, wherein the fingerless glove portion includes a release slit sized to allow the user to remove the hand from the fingerless glove portion.

7. The article of claim 1, further comprising an anti-slip structure extending from the proximal end of the compression sleeve and defining an upper opening sized to receive and sit over at least one of the user's shoulder and the user's head, the anti-slip structure preventing downward migration of the compression sleeve along the limb during physical activity.

8. The article of claim 1, wherein the at least one sweat-management strip is configured to be wrung out by the user without detaching the strip from the compression sleeve.

9. The article of claim 1, wherein the compression sleeve is constructed of a stretchable material selected from the group consisting of spandex, nylon, and combinations thereof.

10. The article of claim 1, wherein the at least one sweat-management strip is provided as a plurality of strips, each strip being independently detachable and replaceable.

11. The article of claim 1, further comprising a sheath formed on an outer surface of the compression sleeve and defining an expandable cavity, the sheath configured to retain a ball.

12. The article of claim 11, wherein the sheath is configured to expand when the ball is positioned within the sheath such that compression applied to the limb is not reduced when the ball is held in the sheath.

13. The article of claim 11, wherein the sheath includes a closure mechanism selected from the group consisting of: a hook and loop fastener, a magnetic structure, an elastic retention mechanism, a zipper, a snap fastener, and combinations thereof.

14. The article of claim 11, wherein the sheath is configured to retain multiple balls.

15. The article of claim 1, further comprising at least one cleaning surface integrated with the compression sleeve and positioned proximate a distal end of the sleeve, the at least one cleaning surface comprising a plurality of debris-removal structures.

16. The article of claim 15, wherein the cleaning surface comprises a removeable strip extending lateral to a longitudinal length of the compression sleeve.

17. The article of claim 15, wherein the cleaning surface is positioned a the distal end of the compression sleeve at a location where a user's knuckles would be when the user's arm is fully inserted into the compression sleeve such that the bending of the user's knuckles causes the debris-removal structures to spread relative to each other facilitating penetration of grooves of a sole of a shoe.

18. The article of claim 15, wherein the debris-removal structures comprises stiff upstanding structures that are configured to engage with grooves in the sole of a shoe.

19. A method for managing perspiration and providing compression therapy during physical activity, comprising:
providing a compression sleeve having a proximal end with an opening for receiving a limb and a distal end with an opening for receiving a hand;
securing at least one detachable sweat-management strip to an exterior surface of the compression sleeve, the at least one sweat-management strip comprising at least one of a moisture-wicking material and an absorbent material;
removably attaching the at least one sweat-management strip at one or more locations along the compression sleeve to facilitate removal, replacement, or repositioning of the sweat-management strip during use;
wherein the compression sleeve and the at least one sweat-management strip collectively provide both compression and enhanced perspiration management for a user during physical activity.

20. The method of claim 19, wherein the at least one sweat-management strip comprises both a moisture-wicking material and an absorbent material.

21. The method of claim 19, further comprising securing the at least one sweat-management strip to the compression sleeve by a hook-and-loop fastener.

22. The method of claim 19, further comprising positioning the at least one sweat-management strip at a location selected from the group consisting of a dorsal radial region, an anterior forearm region, a wrist region, a bicep region, and a palm-knuckle region of the compression sleeve.

23. The method of claim 19, further comprising providing the compression sleeve with a fingerless glove portion at the distal end, the fingerless glove portion including a thumb hole and a plurality of finger holes.

24. The method of claim 23, further comprising providing the fingerless glove portion with a release slit sized to allow the user to remove the hand from the fingerless glove portion.

25. The method of claim 19, further comprising providing an anti-slip structure extending from the proximal end of the compression sleeve and defining an upper opening sized to receive and sit over at least one of the user's shoulder and the user's head, the anti-slip structure preventing downward migration of the compression sleeve along the limb during physical activity.

26. The method of claim 19, further comprising wringing out the at least one sweat-management strip by the user without detaching the strip from the compression sleeve.

27. The method of claim 19, further comprising constructing the compression sleeve of a stretchable material selected from the group consisting of spandex, nylon, and combinations thereof.

28. The method of claim 19, further comprising providing the at least one sweat-management strip as a plurality of strips, each strip being independently detachable and replaceable.

29. The method of claim 19, further comprising forming a sheath on an outer surface of the compression sleeve and defining an expandable cavity, the sheath configured to retain a ball.

30. The method of claim 29, wherein the sheath is configured to expand when the ball is positioned within the sheath such that compression applied to the limb is not reduced when the ball is held in the sheath.

31. The method of claim 29, wherein the sheath includes a closure mechanism selected from the group consisting of: a hook and loop fastener, a magnetic structure, an elastic retention mechanism, a zipper, a snap fastener, and combinations thereof.

32. The method of claim 29, wherein the sheath is configured to retain multiple balls.

33. The method of claim 19, further comprising providing at least one cleaning surface integrated with the compression sleeve and positioned proximate a distal end of the sleeve, the at least one cleaning surface comprising a plurality of debris-removal structures.

34. The method of claim 33, wherein the cleaning surface comprises a removeable strip extending lateral to a longitudinal length of the compression sleeve.

35. The method of claim 33, wherein the cleaning surface is positioned at the distal end of the compression sleeve at a location where a user's knuckles would be when the user's arm is fully inserted into the compression sleeve such that the bending of the user's knuckles causes the debris-removal structures to spread relative to each other facilitating penetration of grooves of a sole of a shoe.

36. The method of claim 33, wherein the debris-removal structures comprise stiff upstanding structures that are configured to engage with grooves in the sole of a shoe.
